SEQUENCE LISTING (59 sequences)

SEQ ID NO: 1 - protein, 24 aa, Bos taurus
GKSGDRGETGPAGPAGPIGPVGAR

SEQ ID NO: 2 - protein, 23 aa, Bos taurus
GKSGDRGETGPAGPAGPIGPVGA

SEQ ID NO: 3 - protein, 21 aa, Bos taurus
SGDRGETGPAGPAGPIGPVGA

SEQ ID NO: 4 - protein, 20 aa, Bos taurus
GKSGDRGETGPAGPAGPIGP

SEQ ID NO: 5 - protein, 21 aa, Bos taurus
GKSGDRGETGPAGPAGPIGPV

SEQ ID NO: 6 - protein, 14 aa, Bos taurus
GADGAPGKDGVRGL

SEQ ID NO: 7 - protein, 20 aa, Bos taurus
GDRGETGPAGPAGPIGPVGA

SEQ ID NO: 8 - protein, 19 aa, Bos taurus
SGDRGETGPAGPAGPIGPV

SEQ ID NO: 9 - protein, 20 aa, Bos taurus
KSGDRGETGPAGPAGPIGPV

SEQ ID NO: 10 - protein, 18 aa, Bos taurus
GDRGETGPAGPAGPIGPV

SEQ ID NO: 11 - protein, 18 aa, Artificial Sequence
  It is synthesized.
GPPGPAGPAGERGEQGPA

SEQ ID NO: 12 - protein, 19 aa, Artificial Sequence
  It is synthesized.
GAPGADGPAGAPGTPGPQG

SEQ ID NO: 13 - protein, 17 aa, Artificial Sequence
  It is synthesized.
DRGETGPAGPAGPIGPV

SEQ ID NO: 14 - protein, 16 aa, Bos taurus
STGISVPGPMGPSGPR

SEQ ID NO: 15 - protein, 18 aa, Bos taurus
RGETGPAGPAGPIGPVGA

SEQ ID NO: 16 - protein, 15 aa, Bos taurus
TGPAGPAGPIGPVGA

SEQ ID NO: 17 - protein, 16 aa, Bos taurus
RGETGPAGPAGPIGPV

SEQ ID NO: 18 - protein, 12 aa, Bos taurus
GERGFPGLPGPS

SEQ ID NO: 19 - protein, 14 aa, Bos taurus
GISVPGPMGPSGPR

SEQ ID NO: 20 - protein, 13 aa, Bos taurus
ISVPGPMGPSGPR

SEQ ID NO: 21 - protein, 13 aa, Bos taurus
TGPAGPAGPIGPV

SEQ ID NO: 22 - protein, 12 aa, Bos taurus
SVPGPMGPSGPR

SEQ ID NO: 23 - protein, 11 aa, Bos taurus
GISVPGPMGPS

SEQ ID NO: 24 - protein, 12 aa, Bos taurus
GPAGPAGPIGPV

SEQ ID NO: 25 - protein, 12 aa, Bos taurus
GPAGPPGPIGNV

SEQ ID NO: 26 - protein, 9 aa, Bos taurus
GPAGPIGPV

SEQ ID NO: 27 - protein, 7 aa, Bos taurus
ISVPGPM

SEQ ID NO: 28 - protein, 12 aa, Bos taurus
GLPGPPGAPGPQ

SEQ ID NO: 29 - protein, 20 aa, Bos taurus
LAGHHGDQGAPGAVGPAGPR

SEQ ID NO: 30 - protein, 19 aa, Bos taurus
GPAGPSGPAGKDGRIGQPG

SEQ ID NO: 31 - protein, 17 aa, Bos taurus
LAGHHGDQGAPGAVGPA

SEQ ID NO: 32 - protein, 20 aa, Bos taurus
GPAGPSGPAGKDGRIGQPGA

SEQ ID NO: 33 - protein, 16 aa, Bos taurus
GPSGPAGKDGRIGQPG

SEQ ID NO: 34 - protein, 18 aa, Bos taurus
GDRGEAGPAGPAGPAGPR

SEQ ID NO: 35 - protein, 9 aa, Bos taurus
GEKGETGLR

SEQ ID NO: 36 - protein, 13 aa, Bos taurus
GPAGKDGRIGQPG

SEQ ID NO: 37 - protein, 13 aa, Bos taurus
LRGPRGDQGPVGR

SEQ ID NO: 38 - protein, 8 aa, Bos taurus
GFDGDFYR

SEQ ID NO: 39 - protein, 14 aa, Bos taurus
ARGSDGSVGPVGPA

SEQ ID NO: 40 - protein, 15 aa, Bos taurus
GDQGAPGAVGPAGPR

SEQ ID NO: 41 - protein, 15 aa, Bos taurus
GARGSDGSVGPVGPA

SEQ ID NO: 42 - protein, 13 aa, Bos taurus
RGSDGSVGPVGPA

SEQ ID NO: 43 - protein, 14 aa, Bos taurus
AVGPAGPRGPAGPS

SEQ ID NO: 44 - protein, 12 aa, Bos taurus
GSDGSVGPVGPA

SEQ ID NO: 45 - protein, 12 aa, Bos taurus
GAAGPTGPIGSR

SEQ ID NO: 46 - protein, 15 aa, Bos taurus
GSDGSVGPVGPAGPI

SEQ ID NO: 47 - protein, 11 aa, Bos taurus
AAGPTGPIGSR

SEQ ID NO: 48 - protein, 12 aa, Bos taurus
GIDGRPGPIGPA

SEQ ID NO: 49 - protein, 9 aa, Bos taurus
GPVGPVGKH

SEQ ID NO: 50 - protein, 9 aa, Bos taurus
GPSGLPGER

SEQ ID NO: 51 - protein, 10 aa, Bos taurus
AGPTGPIGSR

SEQ ID NO: 52 - protein, 9 aa, Bos taurus
GPTGPIGSR

SEQ ID NO: 53 - protein, 13 aa, Bos taurus
VGPAGPRGPAGPS

SEQ ID NO: 54 - protein, 10 aa, Bos taurus
VGPRGPSGPQ

SEQ ID NO: 55 - protein, 12 aa, Bos taurus
GPRGPAGPSGPA

SEQ ID NO: 56 - protein, 12 aa, Bos taurus
VGPAGPRGPAGP

SEQ ID NO: 57 - protein, 12 aa, Bos taurus
GPAGPRGPAGPS

SEQ ID NO: 58 - protein, 10 aa, Bos taurus
VGPAGPRGPA

SEQ ID NO: 59 - protein, 9 aa, Bos taurus
GPAGPAGPR

(19) United States
(12) Patent Application Publication
(10) Pub. No.: US 2022/0308068 A1
(43) Pub. Date: Sep. 29, 2022

(54) BIOMARKER IN OSTEOPOROSIS INTERVENTION THERAPY BY BONE PEPTIDE, SCREENING METHOD AND USE THEREOF

(71) Applicant: Institute of Food Science and Technology, Chinese Academy of Agricultural Sciences, Beijing (CN)

(72) Inventors: Chunhui ZHANG, Beijing (CN); Mengliang YE, Beijing (CN); Yujie GUO, Beijing (CN); Qiankun ZHENG, Zhucheng (CN)

(21) Appl. No.: 17/310,842

(22) PCT Filed: Aug. 20, 2020
(86) PCT No.: PCT/CN2020/110285

§ 371 Date: Aug. 26, 2021

(30) Foreign Application Priority Data
Dec. 13, 2019 (CN) 201911281768.X

(51) Int. Cl. G01N 33/68; G01N 30/06; G01N 30/88

(52) U.S. Cl. CPC G01N 33/6893; G01N 30/06; G01N 30/88; G01N 2030/062; G01N 2030/8813

(57) ABSTRACT

The disclosure discloses a biomarker in osteoporosis intervention therapy by bone peptide, the biomarker including a lipid and lipid-like molecule, an organic acid and its derivative, and/or a neurotransmitter, wherein the lipid and lipid-like molecule includes one or more of taurine, arachidonic acid, 1-palmitoyl-2-hydroxy-sn-glycerol-3-phosphoethanolamine, (4Z, 7Z,10Z,13Z,16Z,19Z)-4,7,10,13,16,19-docosahexaenoic acid, 1-stearoyl-2-hydroxy-sn-glycerol-3-phosphocholine, taurodeoxycholic acid, taurochenodeoxycholate or taurocholic acid. The disclosure discloses a screening method of a biomarker in the anti-osteoporosis activity of bone peptide. The disclosure discloses a use of the biomarker.

### TECHNICAL FIELD

The disclosure relates to the field of nutritional and functional foods, and more specifically to a biomarker in osteoporosis intervention therapy by bone peptide, a screening method of the biomarker in the anti-osteoporosis activity of bone peptide and a use of the biomarker in osteoporosis intervention therapy by bone peptide.

### BACKGROUND

As China entering into aging society, the incidence of osteoporosis among residents is also increasing year by year. At present, there are about 93 million osteoporosis patients in China, and it is predicted that the number of the osteoporosis patients will be close to 200 million by 2050. Osteoporosis is a systemic bone metabolic disease which is characterized by osteopenia, bone microstructural degeneration, and increase of bone fragility. Osteoporosis-induced fractures have increased disability rate and fatality rate, and have become a serious public health problem. In clinical practice, therapeutic drugs for osteoporosis include risedronates, terephthalic acid, alendronic acid, bisphosphonates, zoledronic acid, teriparatide, etc. However, these drugs may induce side effect such as esophagitis, nausea, abdominal pain, and even cancerization of reproductive system, and their applications are limited to a certain extent. Therefore, safe natural alternatives derived from food that can promote bone formation and reverse bone structure damage are drawing more and more attention.

Poultry and livestock bone is rich in collagen. Researches show that collagen peptide can improve regularity and firmness of collagenous fibrillar network, promote orderly deposition of calcium salts, increase bone strength and density, and is an ideal source of potential peptides with anti-osteoporosis activity. At present, some researches have been carried out on the anti-osteoporosis activity and mechanism of bone peptide, but there exists great limitations and one-sidedness in the research level and standard by only observing one or a few typical indicators of bone tissues or organs to evaluate the activity of bone peptide, and it is impossible to systematically and comprehensively reflect and explain the mechanism of bone peptide, so that the development and utilization of bone peptide are greatly limited.

Metabolomics is a systems biotechnology for understanding processes of complex diseases, and is a science about types, quantities and changing laws of metabolites (endogenous metabolites) in an organism after being stimulated or disturbed. Many biological processes of the organism occur at the level of small molecular metabolites. For example, signal release between cells, energy transmission, and communication recognition between cells are completed by mutual regulation of the small molecular metabolites. The research of the organism's changes after being stimulated or disturbed by external disturbances based on the metabolomics level has important prospective significance for revealing the internal mechanism of the organism, whose overall and dynamic concept coincide with the overall research idea of the action of bone peptide multi-components on multiple targets. The research of the anti-osteoporosis activity and mechanism of bone peptide by metabolomics based on a system and an entirety is conducive to objectively and scientifically reflect its dynamic regulation and influence on the organism during an intervention process, and to clarify metabolic networks and target groups regulated by an osteoporosis therapy process of bone peptide.

### SUMMARY

An object of the disclosure is to solve at least the above problems and/or defects, and to provide, at least, the advantages that will be described later.

Another object of the disclosure is to provide a biomarker in osteoporosis intervention therapy by bone peptide.

Another object of the disclosure is to provide a screening method of a biomarker in the anti-osteoporosis activity of bone peptide.

Another object of the disclosure is to provide a use of the biomarker in osteoporosis intervention therapy by bone peptide.

Therefore, the technical solutions provided by the disclosure are as follows.

A biomarker in osteoporosis intervention therapy by bone peptide, the biomarker comprising a lipid and lipid-like molecule, an organic acid and its derivative, and/or a neurotransmitter, wherein the lipid and lipid-like molecule comprises one or more of taurine, arachidonic acid, 1-palmitoyl-2-hydroxy-sn-glycerol-3-phosphoethanolamine, (4Z, 7Z,10Z,13Z,16Z,19Z)-4,7,10,13,16,19-docosahexaenoic acid, 1-stearoyl-2-hydroxy-sn-glycerol-3-phosphocholine, taurodeoxycholic acid, taurochenodeoxycholate or taurocholic acid.

Preferably, for the biomarker in osteoporosis intervention therapy by bone peptide, the organic acid and its derivative comprise D-erythro-sphingosine-1-phosphoric acid and/or L-citrulline.

Preferably, for the biomarker in osteoporosis intervention therapy by bone peptide, the neurotransmitters is serotonin.

A screening method of a biomarker in the anti-osteoporosis activity of bone peptide, comprising the following steps: step one, collecting samples: collecting bone tissues and serum samples from animals treated with bone peptide, wherein the bone tissues comprise left femurs, right femurs and right tibias; step two, determining a content of a serum bone turnover marker by an automatic serum biochemical analyzer, and analyzing the effect of the bone peptide on the content of the serum bone turnover marker; step three, determining biomechanical indexes of the left femurs by a three-point bending test method, and analyzing the effect of the bone peptide on mechanical indexes of femurs; step four, determining biomechanical indexes of the right femurs by a Micro-CT method, and analyzing the effect of the bone peptide on morphologically mechanical indexes of femurs; step five, determining bone microstructure indexes of the right tibias by a H&E staining method, and analyzing the effect of the bone peptide on bone microstructures of tibias of rats; step six, systematically screening and analyzing a differential biomarker (in the serum) in the anti-osteoporosis activity of the bone peptide, as well as its metabolic pathways and regulatory networks based on a non-targeted metabolomics method.

Preferably, for the screening method of a biomarker in the anti-osteoporosis activity of bone peptide, the serum bone turnover marker comprises bone gamma-carboxyglutamic acid containing proteins, bone alkaline phosphatase, procollagen type I N-peptide, tartrate-resistant acid phosphatase, serum C-terminal telopeptide of type I collagen, and urinary deoxypyridinoline; the mechanical indexes comprise fracture load, elastic load, elastic deformation, bending energy and stiffness coefficient of bone; and the morphologically mechanical indexes comprise trabecular bone density (bone density), bone volume fraction (bone volume/total volume), trabecular bone spacing, trabecular bone thickness, trabecular bone number, and cortical bone thickness.

Preferably, for the screening method of a biomarker in the anti-osteoporosis activity of bone peptide, the animals are rats.

Preferably, for the screening method of a biomarker in the anti-osteoporosis activity of bone peptide, in the step one, a treatment process of the animals treated with the bone peptide comprising perfusing an animal with an bone peptide solution, wherein a concentration of the bone peptide solution is 100 mg/kg, 200 mg/kg or 500 mg/kg according to the weight of the animal.

Preferably, for the screening method of a biomarker in the anti-osteoporosis activity of bone peptide, in the step one, the treatment process of the animals treated with bone peptide further comprising automatically collecting urine of the animals with a metabolic cage, wherein the metabolic cage comprises a cage body with a bottom and a metabolite collecting part; the metabolite collecting part being arranged below the cage body and comprising a barrel and a cover mounted on an upper end of a peripheral wall of a first side of the barrel, an upper end of a peripheral wall of a second side of the barrel being provided with a drainage port, a solid-liquid separating part being arranged in the barrel, the solid-liquid separating part comprising an arc-shaped partition plate with a first end fixed with a peripheral wall of the barrel and multi-stage filter plates, which divide an inner space of the barrel into a first accommodating space and a second accommodating space, the multi-stage filter plates being arranged in the second accommodating space along a vertical direction, and the multi-stage filter plates being successively arranged end to end to form a folded-line diversion channel, a depth of a bottom wall of the barrel from the first side to the second side becoming larger, the cover comprising an upper edge bent upwards; a first part of the cover connected to a the barrel being provided with a first through hole, the first end of the arc-shaped partition plate being provided with a second through hole, the second through hole being provided with a filter membrane with 5-20 μm pore size, and the multi-stage filter plates being provided with filter pores whose pore sizes becoming smaller and smaller along the vertical direction from top to bottom and all being larger than the pore size of the filter membrane in the second through hole.

Preferably, for the screening method of a biomarker in the anti-osteoporosis activity of bone peptide, the bone peptide comprises the following peptides: amino acid sequences shown as SEQ ID NO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58 and 59.

A use of the biomarker in scientific research, and intervention therapy or diagnosis of osteoporosis.

The disclosure includes at least the following substantial improvements and beneficial effects:
a. The disclosure discloses systematic evaluation of the anti-osteoporosis activity of bone peptide based on serum bone turnover markers, bone biomechanical indexes and bone morphologically mechanical indexes for the first time, screens biomarkers in the anti-osteoporosis activity of bone peptide by UPLC/Q-TOF-MS technology on the above basis, further clarifies their metabolic pathways and regulatory networks, and comprehensively, efficiently and systematically evaluates the mechanism of the anti-osteoporosis activity of bone peptide from the overall level. The disclosure provides an exemplary research for the activity and function evaluation of natural products (polypeptides), and provides theoretical support for the systematic evaluation of the anti-osteoporosis activity of bone peptide and the development of bone peptide products with biological activity.
b. The disclosure provides one or more of the biomarkers that can specifically indicate serum metabolic fingerprint variation of rats after the improvement of osteoporosis by bovine bone collagen peptide, thereby reflecting the positive effect of the bovine bone peptide on osteoporosis of ovariectomized rats.

Other advantages, objects, and features of the disclosure will be shown in part through the following description, and in part will be understood by those skilled in the art from study and practice of the disclosure.

### BRIEF DESCRIPTION OF THE DRAWINGS

FIG. 1 is a figure showing the effect of bone peptide on the contents of serum bone turnover markers of rats according to the disclosure.

FIG. 2 is a figure showing the effect of bone peptide on biomechanical indexes of left femurs of the rats according to the disclosure.

FIG. 3A is a figure showing three dimensional reconstruction of bone microstructures of the rats according to the disclosure.

FIG. 3B is a figure showing the effect of bone peptide on morphologically mechanical indexes of right femurs of the rats according to the disclosure.

FIG. 4 is a figure showing the effect of bone peptide on bone microstructures (bone tissue pathology) of right tibias of the rats according to the disclosure.

FIG. 5 is a serum metabolic fingerprint analysis figure of the rats after intervention with bone peptide according to the disclosure.

FIG. 6 is a flow figure of a screening method of the biomarker in the anti-osteoporosis activity of bone peptide according to the disclosure.

FIG. 7 is a structural figure of a metabolic cage according to the disclosure.

### DETAILED DESCRIPTION

The disclosure will now be described in further detail with reference to the drawings, in order to enable person skilled in the art to practice with reference to the literal description of the specification.

It should be noted that terms such as “having”, “including” and “comprising” as used herein do not exclude presence or addition of one or more other elements or combinations thereof.

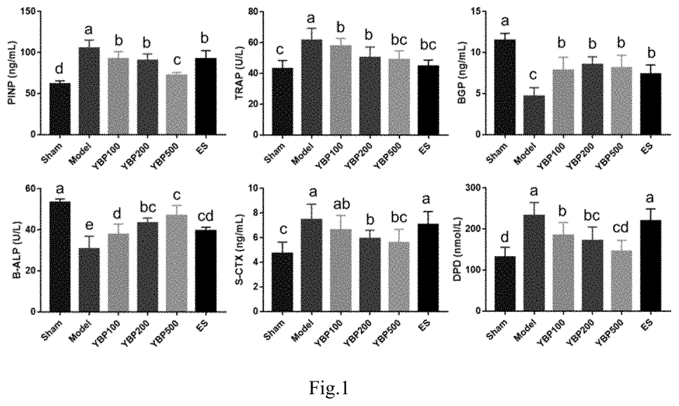

As shown in FIG. 1 to FIG. 7, the disclosure provides a biomarker in the osteoporosis intervention therapy by bone peptide, and the biomarker includes a lipid and lipid-like molecule, an organic acid and its derivative, and/or a neurotransmitter, wherein the lipid and lipid-like molecule includes one or more of taurine, arachidonic acid, 1-palmitoyl-2-hydroxy-sn-glycerol-3-phosphoethanolamine, (4Z, 7Z,10Z,13Z,16Z,19Z)-4,7,10,13,16,19-docosahexaenoic acid, 1-stearoyl-2-hydroxy-sn-glycerol-3-phosphocholine, taurodeoxycholic acid, taurochenodeoxycholate or taurocholic acid. In the above solution, it is preferred that the organic acid and its derivative include D-erythro-sphingosine-1-phosphoric acid and/or L-citrulline. In the above solution, it is preferred that the neurotransmitters is serotonin.

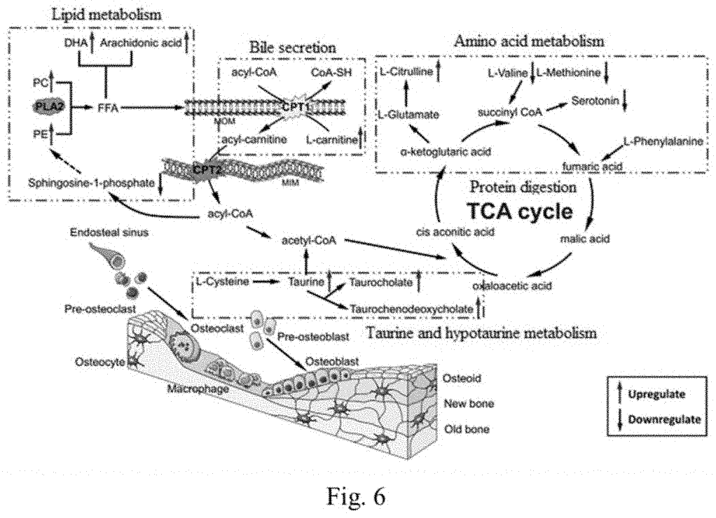

As shown in FIG. 6, the disclosure also provides a screening method of a biomarker in the anti-osteoporosis activity of bone peptide, include the following steps:

step one, collecting samples: collecting bone tissues and serum samples from animals treated with bone peptide, wherein the bone tissues include left femurs, right femurs and right tibias;

step two, determining a content of a serum bone turnover marker by an automatic serum biochemical analyzer, and analyzing the effect of the bone peptide on the content of the serum bone turnover marker;

step three, determining biomechanical indexes of the left femurs by a three-point bending test method, and analyzing the effect of the bone peptide on mechanical indexes of femurs;

step four, determining biomechanical indexes of the right femurs by a Micro-CT method, and analyzing the effect of the bone peptide on morphologically mechanical indexes of femurs;

step five, determining bone microstructure indexes of the right tibias by a H&E staining method, and analyzing the effect of the bone peptide on bone microstructures of tibias of rats;

step six, systematically screening and analyzing a differential biomarker (in the serum) in the anti-osteoporosis activity of the bone peptide, as well as its metabolic pathways and regulatory networks based on a non-targeted metabolomics method.

In the above solution, it is preferred that the serum bone turnover marker includes bone gamma-carboxyglutamic acid containing proteins, bone alkaline phosphatase, procollagen type I N-peptide, tartrate-resistant acid phosphatase, serum C-terminal telopeptide of type I collagen, and urinary deoxypyridinoline; the mechanical indexes include fracture load, elastic load, elastic deformation, bending energy and stiffness coefficient of bone; and the morphologically mechanical indexes include trabecular bone density (bone density), bone volume fraction (bone volume/total volume), trabecular bone spacing, trabecular bone thickness, trabecular bone number, and cortical bone thickness. In the above solution, it is preferred that the animals are rats.

In the above solution, it is preferred that a treatment process of the animals treated with the bone peptide in the step one includes perfusing an animal with an bone peptide solution, wherein a concentration of the bone peptide solution is 100 mg/kg, 200 mg/kg or 500 mg/kg according to the weight of the animal.

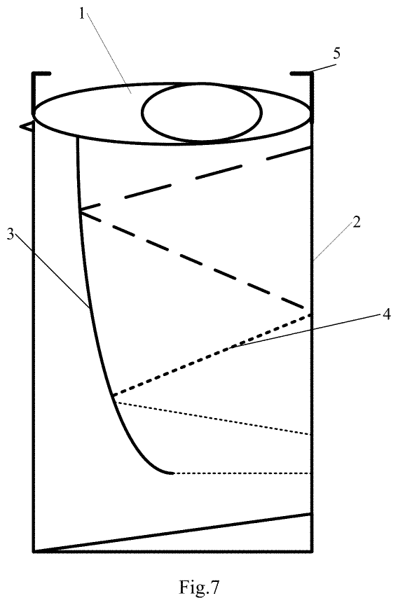

In the above solution, it is preferred that the treatment process of the animals treated with bone peptide in the step one further includes automatically collecting urine of the animals with a metabolic cage. As shown in FIG. 7, the metabolic cage comprises a cage body with a bottom and a metabolite collecting part. The metabolite collecting part is arranged below the cage body and comprises a barrel 1 and a cover 2 mounted on an upper end of a peripheral wall of a first side of the barrel, an upper end of a peripheral wall of a second side of the barrel is provided with a drainage port, and a solid-liquid separating part is arranged in the barrel. The solid-liquid separating part comprises an arc-shaped partition plate 3 with a first end fixed with a peripheral wall of the barrel and multi-stage filter plates 4, which divide an inner space of the barrel into a first accommodating space and a second accommodating space. The multi-stage filter plates are arranged in the second accommodating space along a vertical direction, and the multi-stage filter plates are successively arranged end to end to form a folded-line diversion channel. A depth of a bottom wall of the barrel from the first side to the second side becomes larger. The cover comprises an upper edge 5 bent upwards; a first part of the cover connected to a the barrel being provided with a first through hole. The first end of the arc-shaped partition plate is provided with a second through hole. The second through hole is provided with a filter membrane with 5-20 μm pore size, and the multi-stage filter plates are provided with filter pores whose pore sizes becoming smaller and smaller along the vertical direction from top to bottom and all are larger than the pore size of the filter membrane in the second through hole.

In the above solution, it is preferred that the bone peptide includes the following peptides: amino acid sequences shown as SEQ ID NO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58 and 59.

The bone peptide includes the following peptides: amino acid sequences shown as SEQ ID NO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58 and 59.

A use of the biomarker or the bone peptide in scientific research, and intervention therapy or diagnosis of osteoporosis.

In order to enable person skilled in the art to better understand the technical solutions of the disclosure, the disclosure will now be described with bovine bone collagen peptide (bone peptide) that is prepared by the inventors and has a significant osteoblast proliferation activity in vitro as a research object.

A screening method of the biomarker in the anti-osteoporosis activity of the bone peptide includes the following main steps:

step one, collecting samples: collecting bone tissues (left femurs, right femurs and a right tibias) and serum samples from rats treated with bone peptide;

step two, determining a content of a serum bone turnover marker by an automatic serum biochemical analyzer, and analyzing the effect of the bone peptide on the content of the serum bone turnover marker of the rat;

step three, determining biomechanical indexes of the left femurs of rats by a three-point bending test method, and analyzing the effect of bone peptide on mechanical indexes of femurs of rats;

step four, determining biomechanical indexes of the right femurs of rats by a Micro-CT method, and analyzing the effect of the bone peptide on morphologically mechanical indexes of femurs of rats;

step five, determining bone microstructure indexes of the right tibias of rats by a H&E staining method, and analyzing the effect of the bone peptide on bone microstructures of tibias of rats;

step six, systematically screening and analyzing a differential biomarker (in the serum) of anti-osteoporosis activity of the bone peptide, as well as its metabolic pathways and regulatory networks based on a non-targeted metabolomics method.

In the screening method of the biomarkers in the anti-osteoporosis activity of the bone peptide, the bone tissues (left femurs, right femurs and a right tibias) and serum samples are from rats fed by the inventors, and the specific steps are as follows.

1, the construction of ovariectomized rats models: SD rats are kept in a clean environment under a controlled room temperature at 25±2° C. and alternates 12/12 light and dark every day, and the SD rats feed freely. After one week adaptive feeding, randomly select 8 female rats, anesthetize them with 1% (v/v) pentobarbital sodium (40 mg/kg BW), and remove a little fat near ovaries. The remaining 40 rats are ovariectomized after being anesthetized with pentobarbital sodium. The recovery situation is observed in a 4-week recovery period, and their weight changes are detected. These are prepared by the applicant in advance. (any research about metabolic fingerprint changes of ovariectomized osteoporotic rats after intervention with bone peptide have not been reported according to the existing literature).

2, animal grouping and samples collection: the 8 female rats with a little fat near ovaries removed are selected as a sham-operated group, the ovariectomized 40 rats are randomly divided into 5 groups with 8 rats in each group, and are named by a negative control group, a positive control group, a low-concentration treatment group, a medium-concentration treatment group and a high-concentration treatment group. The rats are perfused by different solutions of bovine bone peptide (diluted by ultrapure water, and sterilized) with concentration of 100 mg/kg, 200 mg/kg, and 500 mg/kg according to the weights of the rats. The rats in the negative control group are perfused with equal volume of ultrapure sterile water (a perfusing volume is generally 1-2 mL/100 g BW), and the rats in the positive control group are perfused with 50 μg/kg of 17β-estradiol (ES). Observe weight changes of the rats and measure the weights every two weeks.

Urine is automatically collected with a metabolic cage for 12 hours every 4 weeks (the urine is collected as much as possible under the premise of ensuring normal signs of the rats). 1 mmol/L NaN₃ solution is added into collected urine as a preservative, the collected urine is placed in a centrifuge at 4° C. and centrifuged at a speed of 10000×g for 10 minutes, and the supernatant is collected, distributed and stored in a refrigerator at −80° C. for determining. The rats are fasted for 12 hours at 4th week, 8th week, and 12th week. Fasted rats are anesthetized by intraperitoneal injection of pentobarbital sodium (40 mg/kg BW) with a volume concentration of 1%, and are subjected to blood collection from the abdominal aorta (the blood is collected as much as possible under the premise of ensuring normal signs of the rats). Collected blood is placed at 4° C. for 3 h and is centrifuged (5000 rpm) for 10 minutes. Upper serum is collected (collecting 2 mL of blood, separating into 4 tubes after separating the serum), is divided into 0.5 mL EP tubes, and stored in a refrigerator at −80° C. for later use. The metabolic cage comprises a cage body with a bottom and a metabolite collecting part. The metabolite collecting part is arranged below the cage body and comprises a barrel 1 and a cover 2 mounted on an upper end of a peripheral wall of a first side of the barrel, an upper end of a peripheral wall of a second side of the barrel is provided with a drainage port, and a solid-liquid separating part is arranged in the barrel. The solid-liquid separating part comprises an arc-shaped partition plate 3 with a first end fixed with a peripheral wall of the barrel and multi-stage filter plates 4, which divide an inner space of the barrel into a first accommodating space and a second accommodating space. The multi-stage filter plates are arranged in the second accommodating space along a vertical direction, and the multi-stage filter plates are successively arranged end to end to form a folded-line diversion channel. A depth of a bottom wall of the barrel from the first side to the second side becomes larger. The cover comprises an upper edge 5 bent upwards; and a first part of the cover connected to a the barrel being provided with a first through hole. The first end of the arc-shaped partition plate is provided with a second through hole. The second through hole is provided with a filter membrane with a 5-20 μm pore size, and the multi-stage filter plates are provided with filter pores whose pore sizes become smaller and smaller along the vertical direction from top to bottom and all are larger than the pore size of the filter membrane in the second through hole.

After perfusing experiment, the rats are sacrificed in accordance with the animal welfare operating procedures, femurs and tibias on both sides are taken, and soft tissues such as muscle and fascia attached to bone tissues are removed. The right tibias are subjected to paraffin-embedded H&E staining treatment after fixing in a phosphate-formalin buffer for 24 hours for morphometric analysis of the tibias of the rats. Left femurs and right femurs is soaked with normal saline, washed repeatedly for 3 times, wrapped with medical gauzes (pre-soaked with normal saline) and tinfoil, and then stored in a −20° C. refrigerator for trabecular bone microstructure (micro-CT scanning) and mechanical strength tests of bone biomechanical indexes (three-point bending test).

In the screening method of the biomarkers in the anti-osteoporosis activity of bone peptide, the specific implementation steps of determining the content of a bone turnover marker by an automatic serum biochemical analyzer are as follows: anesthetizing the rats, collecting blood from abdominal aorta, standing at room temperature for 10 minutes, centrifuging at 10000×g speed for 10 minutes, collecting upper serum, and storing in a −80° C. refrigerator or directly determining serum biochemical indexes by the automatic serum biochemical analyzer (the bone turnover markers are determined by a kit method). The serum bone turnover markers include bone gamma-carboxyglutamic acid containing proteins (BGP), bone alkaline phosphatase (B-ALP), procollagen type I N-peptide (PINP), tartrate-resistant acid phosphatase (TRAP), serum C-terminal telopeptide of type I collagen (S-CTX), and urinary deoxypyridinoline (DPD).

In the screening method of the biomarkers in the anti-osteoporosis activity of bone peptide, the specific implementation steps of determining biomechanical indexes of the left femurs of the rats by the three-point bending test method are as follows: the three-point bending test is a common method to determine bone biomechanical indexes for reflecting bone strength changes, performing room-temperature thawing of the left femurs of the rats frozen at −20° C., rinsing and soaking with normal saline; putting the bone tissue onto a LLOYD universal material testing machine with parameters: a span (L) of 10 mm and a loading speed of 2 mm/min, and automatically recording fracture load (Fd), elastic load (Ed), elastic deformation (En), bending energy (Be) and stiffness coefficient (Sc) with software.

In the screening method of the biomarkers in the anti-osteoporosis activity of bone peptide, the specific implementation steps of determining biomechanical indexes of the right femurs of the rats by the Micro-CT method are as follows: determining femur microstructures by Micro Computed Tomography (Micro-CT, Inveon-type, SIEMENS, Germany) with scanning parameters: a span voltage of 80 kV, a scanning current of 500 μA, and a scanning thickness (resolution) of 14.93 Region of interest (ROI) of femurs starts from 1 mm below a bone tissue growth-plate. Scan the layers downward and sequentially. Select the bone tissue with a thickness of 100 layers as cancellous ROI for three-dimensional reconstruction to obtain a visualized 3D image. The obtained scanning data are subjected to morphometric calculation of femur tissues using Inveon Research Workplace software (SIEMENS, Germany). The biomechanical indexes mainly include trabecular bone density (bone density), bone volume fraction (bone volume/total volume), trabecular bone spacing, trabecular bone thickness, trabecular bone number, and cortical bone thickness.

| index name | abbreviation | unit | connotation |
|---|---|---|---|
| bone density | Tb.BMD | g/cm³ | mineral density in bone tissues |
| bone volume | BV/TV | % | a ratio of bone tissue volume to |
| fraction (bone | | | tissue volume that can directly |
| volume/total | | | reflect bone mass changes |
| volume) | | | |
| trabecular | Tb.Th | μm | average thickness of trabecular |
| bone thickness | | | bone in ROI |
| trabecular | Tb.N | 1/mm | average number of intersections |
| bone number | | | between bone tissue and non- |
| | | | bone tissue with mm unit in |
| | | | ROI |
| trabecular | Tb.SP | μm | average width of medullary |
| bone spacing | | | cavity between trabecular bones |
| Cortical | Cw.T | mm | average thickness of cortical |
| bone thickness | | | bone in ROI |

In the screening method of the biomarkers in the anti-osteoporosis activity of bone peptide, the specific implementation steps of determining microstructural indexes of the right tibias of the rats by the H&E staining method are as follows: fixing the right tibias of the rats with 10% formalin for 48 h, decalcifying with EDTA for 30 d, embedding the tissue in paraffin, cutting it into 3 mm slices, staining it with a hematoxylin and eosin (H&E) solution, and performing histological observation of tibias under an automatically digital scanning system (KF-PRO-120, Ningbo Jiangfeng Bioinformatics Technology Co., Ltd.) for the slices.

In the screening method of the biomarkers in the anti-osteoporosis activity of bone peptide, the specific implementation steps of selecting and analyzing biomarkers (in the serum) in the anti-osteoporosis activity of the bone peptide, as well as their metabolic pathways and regulatory networks based on the non-targeted metabolomics method are as follows:
a. Pretreatment method for animal serum samples: pretreatment of quality control (QC) samples: accurately pipetting an appropriate amount of samples and mixing in equal ratio to prepare QC samples. The QC samples are mainly used to monitor, confirm the status and stability of an equipment, balance a High Performance Liquid Chromatography-Mass Spectrometry (HPLC-MS) analysis system, and comprehensively evaluate the stability of system during the entire experiment process. Take each serum sample, slowly thaw at 4° C., and then divide it into 100 μL/tube. Besides, take 100 μL of each sample, mix and prepare a QC sample. Add 400 μL of pre-cooled methanol/acetonitrile (v/v, 1:1) solution to every 100 μL sample at 4° C., shake and mix it, stand at −20° C. for 10 min, centrifuge it at a 14000×g speed and 4° C. for 15 min, collect the supernatant, freeze-dry it, and store it in a −80° C. refrigerator for later use.
b. Chromatography-Mass Spectrometry condition analysis: the serum samples of the rats are separated by Agilent 1290 Infinity ultra-high pressure liquid chromatography (UPLC) (a chromatography column is a HILIC column). The chromatography parameters are set as follows: a column temperature: 25° C.; a flow rate: 0.3 mL/min; an injection volume: 2 μL; a mobile phase A: (water+25 mM ammonium acetate+25 mM ammonia), a mobile phase B (acetonitrile).

A gradient elution procedure is as follows:

| index name | abbreviation | unit | connotation |
|---|---|---|---|
| bone density | Tb.BMD | g/cm³ | mineral density in bone tissues |
| bone volume | BV/TV | % | a ratio of bone tissue volume to |
| fraction (bone | | | tissue volume that can directly |
| volume/total | | | reflect bone mass changes |
| volume) | | | |
| trabecular | Tb.Th | μm | average thickness of trabecular |
| bone thickness | | | bone in ROI |
| trabecular | Tb.N | 1/mm | average number of intersections |
| bone number | | | between bone tissue and non- |
| | | | bone tissue with mm unit in |
| | | | ROI |
| trabecular | Tb.SP | μm | average width of medullary |
| bone spacing | | | cavity between trabecular bones |
| Cortical | Cw.T | mm | average thickness of cortical |
| bone thickness | | | bone in ROI |

A positive ion mode and negative ion mode of electrospray ionization (ESI) are used for detection, and the mass spectrometry analysis of the serum samples of the rats is carried by Agilent 6550 mass spectrometer after separating the samples by UPLC. The parameters of ESI are set as follows: a dissolvent gas temperature: 250° C., a flow rate: 16 L/min; a cone-hole gas temperature: 400° C., a flow rate: 12 L/min; a capillary voltage: 3.0 kV; fragment: 175 V; a mass range: 50-1200; an acquisition rate, 4 Hz; a cycle time: 250 ms.

After the serum samples are detected, metabolites detected in the serum samples are identified by a AB Triple TOF 6600 mass spectrometer, and primary and secondary spectra of the QC samples are collected, and collected data are subjected to structural identification of metabolites by self-built MetDDA and LipDDA methods, respectively.

ESI parameters are set as follows:

| Name | Parameters | |
|---|---|---|
| Ion Source Gas1 (Gas1) | 40 | |
| Ion Source Gas2 (Gas2) | 80 | |
| Curtain gas (CUR) | 30 | |
| Ionization source temperature | 650° | C. |
| IonSapary Voltage Floating (ISVF) | ±5000 | V |
| Collision voltage | 50 | V |
| Exclude isotopes | 4 | Da |
| Candidate ions to monitor per cycle | 10 | |
| Mass range | 50-300, 290-600, | |
| | 590-900, 890-1200 | |
| Declustering potential (DP) | ±60 | V |

3. Treatment method of chromatography-mass spectrometry data: primary raw data of the serum samples of rats detected by Agilent are subjected to format conversion (mzXML) by MSconventer, chromatographic peaks and retention time of detected metabolites are calibrated by XCMS program, and peak areas of the detected metabolites by chromatography are accurately extracted, and a minfrac parameter is set to 0.5. The detected metabolites of the serum samples of rats are accurately matched with identification results according to two parameters: charge-mass ratio (m/z±30 ppm) and retention time (RT, ±60 s). Extracted chromatography-mass spectrometry data are subjected to standardization and normalization by a SVR method, and multidimensional statistical data analysises (PCA, OPLS-DA, t-test, variation multiple analysis, R language volcano plot analysis) are performed by SIMCA-P 14.1 (Umetrics, Sweden).

Results and Analysis
a. Determining a content of a serum bone turnover marker by the automatic serum biochemical analyzer is used for analyzing the effect of bovine bone peptide (YBP) on the content of the serum bone turnover marker of the rat.

Serum bone turnover markers (BTMs) are self-synthesized and catabolized products of bone tissues in an organism, and also referred to as bone turnover markers, which can be divided into bone resorption markers and bone formation markers according to effect types. The bone resorption markers are mainly used to reflect osteoclast activity and bone resorption level, and the bone formation markers are used to reflect osteoblast and bone formation status. The determination of the BTMs has great potential for early screening of osteoporosis, assessing fracture risk, and monitoring therapeutic effect of patients after taking therapeutic drugs. Normally, three high-sensitive indicators such as BGP, B-ALP and PINP are used to reflect the bone formation status, and other three high-sensitive indicators such as TRAP, S-CTX and DPD are used to reflect bone resorption status, and then to judge dynamic changes of bone metabolism of whole organism.

The determination results of serum BTMs of the SD rats in a sham-operated group (Sham group), the negative control group-the model group (Model group), the positive control group (ES group), a low-concentration bovine bone peptide treatment group (YBP100 group), a medium-concentration bovine bone peptide treatment group (YBP200 group) and a high-concentration bovine bone peptide treatment group (YBP500 group) are shown in FIG. 1. The results show that the contents of B-ALP and BGP in the Model group are significantly lower than those in the Sham group (P is less than 0.05), indicating that ovariectomized osteoporosis rat model is successfully constructed, which is consistent with a research result of Wang Rong et al. (2017). Compared with the Model group, the contents of B-ALP and BGP in treatment groups and the ES group are significantly increased (P is less than 0.05). The treatment groups treated with different-concentration bovine bone peptide exist a certain concentration effect. The contents of B-ALP in the YBP200 group, YBP500 group and the ES group have no significant difference, and the contents of BGP in the treatment groups treated with different-concentration bovine bone peptide and the ES group have no significant difference (P is more than 0.05). The contents of PINP, TRAP, S-CTX and DPD in the Model group are significantly lower than those in the Sham group (P is less than 0.05), and the above four biochemical indexes in the treatment groups and the ES group are on a decline trend, which shows that bovine bone peptide and estradiol have the same effect for improving osteoporosis-related bone turnover markers. It should be noted that the contents of PINP, S-CTX and DPD in the YBP500 group are significantly lower than those in the ES group (P is less than 0.05), which shows that the improvement effect of bovine bone peptide on these three serum biochemical indexes is stronger than that of the ES group treated with estradiol.

2. Determining biomechanical indexes of the left femurs of the rats by the three-point bending test method is used for analyzing the effect of bovine bone peptide on the mechanical indexes of femurs of the rats.

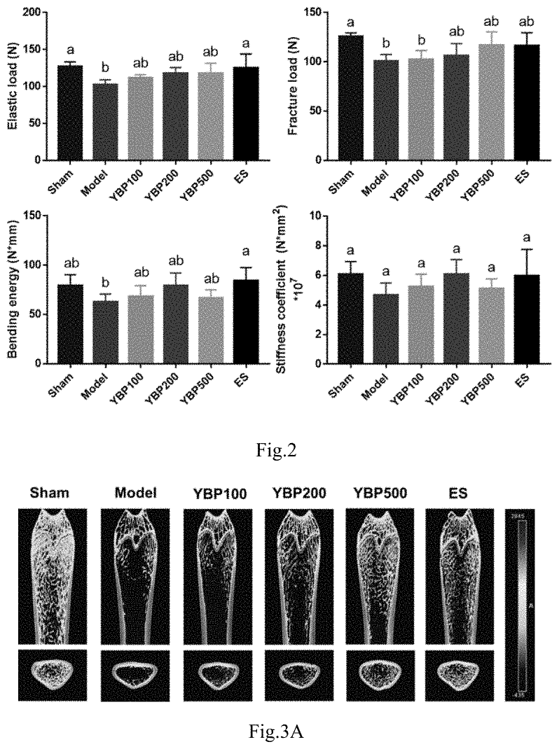

Take the leftfemurs of the rats in the Sham group, Model group, ES group, YBP100 group, YBP200 group and YBP500 group, and changes of the biomechanical indexes (FIG. 2) of the left femurs of the rats in the Sham group, Model group, ES group, YBP100 group, YBP200 group and YBP500 group are determined by the three-point bending test method. The results show that the elastic load (Ed), fracture load (Fd), bending energy (Be) and stiffness coefficient (Sc) in the Model group are lower than those in the Sham group, with the elastic load (Ed) and the fracture load (Fd) being significantly lower, (P is less than 0.05), which indicates that the ovariectomized osteoporosis rat model is successfully constructed. Ed and Fd in the treatments groups (YBP100 group, YBP200 group and YBP500 group) and the ES group are on a rise trend. However, the treatment groups treated with different-concentration bovine bone peptide have no significant difference (P is more than 0.05), and the treatment groups and ES group have no significant difference (P is more than 0.05). Besides, although Be and Sc in the treatment groups (YBP100 group, YBP200 group and YBP500 group) and the ES group exist a certain concentration effect, there was no significant difference among these groups.

3. Determining biomechanical indexes of the right femurs of the rats by the Micro-CT method is used for analyzing the effect of bovine bone peptide on the morphologically mechanical indexes of femurs of the rats.

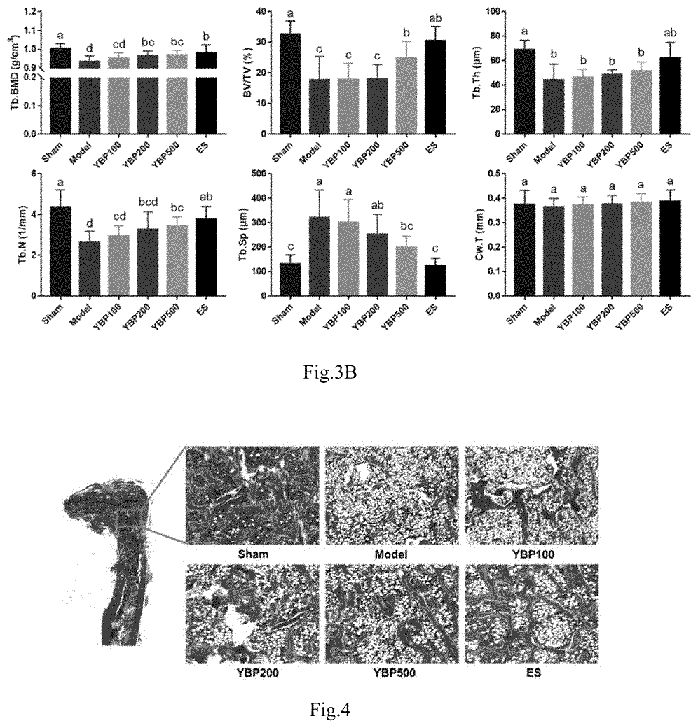

Three dimensional reconstruction (FIG. 3A) of bone microstructures of femurs of the SD rats in the Sham group, Model group, ES group, YBP100 group, YBP200 group and YBP500 group are performed by the Micro-CT method. The results show that trabecular bone density and trabecular bone number in the Model group are significantly lower than those in the Sham group (P is less than 0.05), which indicates that the ovariectomized osteoporosis rat model is successfully constructed. The osteoporosis of the rats is improved to a certain extent after being treated with bovine bone peptide and estradiol. Bovine bone peptide shows a certain dose-effect relationship in improving osteoporosis of the rats, and the effect on improving osteoporosis of the rats is gradually increasing with the increase of the concentration of bovine bone peptide.

The indexes of trabecular bone density (Tb. BMD), bone volume fraction (bone volume/total volume, BV/TV), trabecular bone thickness (Tb.Th), trabecular bone number (Tb.N), trabecular bone spacing (Tb.Sp) and cortical bone thickness (Cw.T) of the femurs of the SD rats in the Sham group, Model group, ES group, YBP100 group, YBP200 group and YBP500 group are determined (FIG. 3B). The results show that Tb.BMD, BV/TV, Tb.Th, and Tb.N of the femurs of the rats in the Model group are significantly lower than those of the Sham group (P is less than 0.05), and Tb.Sp is on a significant rise trend (P is less than 0.05), which indicates that the ovariectomized osteoporosis rat model is successfully constructed. Compared with the Model group, Tb.BMD, BV/TV, Tb.Th, and Tb.N of the femurs of the rats treated with bovine bone peptide and estradiol are on a rise trend, but Tb.BMD, Tb.Th, Tb.N of the femurs of the rats in the YBP100 group, YBP200 group and YBP500 group have no significant difference.

Especially, it is found that Tb.BMD, BV/TV, and Tb.N of the femurs of the rats in the YBP500 group can be significantly increased, so that the bovine bone peptide has a potential improvement effect on osteoporosis of the rats.

Particularly, the H&E staining results show that trabecular bone structures of the rats in the Model group after 12 weeks of intervention treatment are significantly less than those of the Sham group. Besides, trabecular bone area of the rats after the intervention treatment with bovine bone peptide and estradiol are significantly increased, trabecular bone connection is tighter, trabecular bone width becomes wider, and the trabecular bone spacing becomes smaller (FIG. 4). In short, bovine bone peptide can significantly improve bone microstructures and maintain bone mass in the ovariectomized rats, especially the YBP500 group.

4. Systematically screening and analyzing a differential biomarker (in the serum) in the anti-osteoporosis activity of the bovine bone peptide based on the non-targeted metabolomics method, as well as its metabolic pathways and regulatory networks.

Experimental data analysis of quality control: system stability of the experimental instrument is comprehensively evaluated by two methods of spectrum comparison of the QC samples and PCA analysis. The UHPLC-Q-TOF MS total ion chromatogram of 8 QC samples are subjected to chromatographic peak overlap comparative analysis. The results show that the response value and retention time of chromatographic peaks of 8 QC samples are basically the same, which indicates instrument and equipment state is stable during the whole experiment process, the degree of variation caused by method error is small, and can meet the needs of the experiment.

Ion peaks of the metabolites are extracted by XCMS software. The number of the ion peaks are 9676 (positive ion) and 5584 (negative ion), respectively. After Pareto-scaling, the serum of the rats in different groups and the peaks extracted from the QC samples are subjected to principal component analysis, and the results show that 8 QC samples can be closely clustered in a certain area in the positive and negative ion scanning modes, which indicates that the equipment conditions in the experiment have good repeatability and stability.

Analysis of an overall sample Hotellings T2 is usually used to detect whether there are outliers, and the results show that all samples in the experiment are within a 99% confidence interval under the negative ion mode, which indicates that the equipments is stable and experimental data are real and reliable.

The QC samples are subjected to Pearson correlation analysis. The horizontal coordinate and the vertical coordinate in the figure represent logarithm value of strength value, respectively, and a correlation coefficient greater than 0.9 generally indicates a nice correlativity. The results show that the correlation coefficient of the QC samples in the experiment are all greater than 0.9, which meets the requirements of subsequent test analysis and determination.

The QC samples are subjected to maleimide-cyclohexane-1-carboxylate (MCC) analysis, which can produce a multivariable control chart based on a combination of all X variables, can display measured experimental data in real time, and can monitor changes during the experiment process. Each point in the MCC analysis represents a QC sample. Normally, most points are within a control range and fluctuate up and down on the X axis. Generally, it is reasonable within a range of positive and negative three standard deviations, which indicates that the equipments have low volatility. The results show that experimental conditions are relatively stable and monitored data can be used for subsequent analysis.

5. Analysis and screening of potential biomarkers by Multivariate statistics

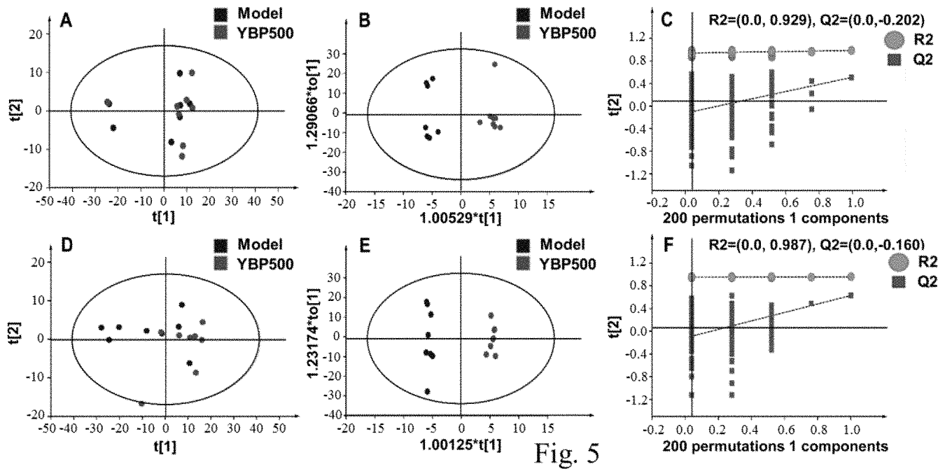

Principal component analysis (PCA) is an unsupervised data statistical analysis method. By PCA, all identified metabolites are subjected to linear arrangement and combination again, and then form a new set of comprehensive statistical variables from which several comprehensive variables that can reflect vertical and horizontal information of the original variables as fully as possible are selected, so as to achieve the purpose of reducing dimensionality and accurate analysis. Normally, the principal component analysis of serum metabolites of the rats in different groups can also overally reflect the degree of variation of the serum samples of the rats between groups and within groups. In summary, PCA can be used to accurately classify samples based on the differences in metabolic fingerprints of serum metabolites of the rats in different groups, thereby realizing rapid mining of massive data. Metabolites of the serum samples of the SD rats in the Sham group, Model group, ES group, YBP100 group, YBP200 group, and YBP500 group are subjected to PCA (FIG. 5), and the results show that metabolites of the serum samples of the rats in the Sham group, Model group, and ES group have great difference, and the distribution of the metabolites shows a certain regularity. Except for individual samples, the metabolites of the serum samples of the rats in the above 6 groups can be classified by PCA. There are many overlapping areas between the treatment groups with different concentrations of bovine bone peptide, which indicates that there is a certain crossover in their metabolic fingerprints. It is noted that their metabolic fingerprints tend to move closer to the metabolic fingerprints of the ES group (with estradiol) and Sham group as the concentration of bovine bone peptide increases. Therefore, the YBP 500 group is selected as a research object in the disclosure, and is used for systematical analysis of the mechanism of anti-osteoporotic activity of bovine bone peptide.

Based on the above analysis, PCA is performed on the metabolites of the serum samples of the rats in the YBP500 group and Model group (Table 1 and FIG. 5). A first principal component PC1 (t[1]) represents the horizontal ordinate of a PCA model, and a second principal component PC2 (t[2]) represents the vertical ordinate of the PCA model. The parameters of principal components model mainly refer to the value of R2X, and R2X closer to 1 indicates that the PCA model is more stable and reliable. In the PCA of the metabolites of the serum samples of the rats in the YBP500 group and Model group, a PCA scoring figure is shown in FIG. 5, where A represents the number of principal components in the PCA model; R2X represents the interpretation rate of the model to X variable; and Q2 represents the predictive ability of the principal components model.

TABLE 1

| sample group | A | R2X (cum) | Q2 (cum) |
|---|---|---|---|
| QC | 5 | 0.564 | 0.298 |
| YBP500-Model | 2 | 0.447 | 0.103 |

Orthogonal projections to latent structures discriminant analysis (OPLS-DA) is a supervised data statistical discriminant analysis method, which can effectively adopts a projections to latent structures regression method to establish a relationship model between two of the expression of serum metabolites of the rats, sample group, and category (Sham, Model, ES, YBP100, YBP200, YBP500), so as to rapidly realize accurate prediction of sample group and category, effectively filter out noise that is not related to classification information, and improve analytical ability of the model and reliability and effectiveness of data classification. OPLS-DA models (Table 2 and FIG. 5) of serum samples of the rats in the YBP500 group and Model group are established. In a OPLS-DA scoring figure, there are two principal components, namely a predictive principal component (uniqueness, t[1]) and orthogonal principal components (there may be multiple). The OPLS-DA model can usually reflect maximization difference between groups on the predicted principal component t[1], so the variation between groups can be directly distinguished from the horizontal ordinate (t[1]), and the variation within groups is reflected on the vertical ordinate (orthogonal principal components).

TABLE 2

| | | R2X | R2Y | Q2 | R2 | Q2 |
|---|---|---|---|---|---|---|
| Sample group | A | (cum) | (cum) | (cum) | intercept | intercept |
| YBP500-Model | 6 | 0.692 | 1.000 | 0.458 | 0.999 | 0.0598 |

The established OPLS-DA model of the serum samples of the rats in the YBP500 group and Model group is verified (multiple circular interactions). Model evaluation parameters (R2Y, Q2) are shown in Table 2. The closer the R2Y value and the Q2 value are to 1, the more realistic and reliable the established model is. Generally, Q2 more than 0.5 indicates that the established model is stable and reliable, Q2 more than 0.3 and no more than 0.5 indicates that the established model is stable, and Q2 less than 0.3 indicates that the reliability of the established model is low; where A represents the number of principal components; R2X represents interpretation rates of the established model to X variable; R2Y represents interpretation rates of the established model to Y variable, and Q2 represents the predictive ability of the established model.

Variable importance for the projection (VIP) obtained from OPLS-DA model analysis is used to measure and evaluate influence strength and interpretation ability of the expression pattern of metabolites to the classification of the serum samples of the rats in the YBP500 group and the Model group. VIP greater than 1 is selected as a screening standard in the disclosure, differential metabolites between the YBP500 group and the Model group are preliminarily screened, and then whether there is a significant difference between metabolites (between groups) is subjected to rational verification based on the results of the univariate statistical analysis. Generally, the metabolites with VIP value greater than 1 and P-value in the univariate statistical analysis less than 0.05 are identified as significantly different potential biomarkers, and the compounds with VIP value greater than 1 and P-value within 0.05-0.1 are identified as different metabolites.

The identified and screened 41 kinds of significantly different metabolites are subjected to database search and comparison. The 41 kinds of potential biomarkers comprise 14 organic acids and their derivatives (isoleucine-alanine, L-methionine, L-pipecolic acid, L-valine, L-tyrosine, N2-acetyl-L-ornithine, NG, NG-dimethyl-L-arginine, proline-alanine, proline-serine, Ergothioneine, L-citrulline, leucine-glycine, diaminoheptanoic acid, erucic amide, and DL-indole-3-lactic acid), 11 lipid and lipid-like molecule (taurine, taurodeoxycholic acid, 1-stearoyl-2-hydroxy-sn-glycerol-3-phosphocholine, taurocholic acid, (4Z, 7Z,10Z,13Z,16Z,19Z)-4,7,10,13,16,19-docosahexaenoic acid, Taurochenodeoxycholate, Tauroursodeoxycholic acid, Thioetheramide-PC, D-erythro-sphingosine-1-phosphate, arachidonic acid, and 1-palmitoyl-2-hydroxy-sn-glycerol-3-phosphoethanolamine), 3 organic nitrogen compounds (L-carnitine, diethanolamine, and hydroxyquinoline), 3 organic heterocyclic compounds (bilirubin, serotonin, and 4-pyridoxic acid), 4 carbohydrates and carbohydrate polyketides (D-fructose, D-tagatose, daidzein, and 4-hydroxycinnamic acid), 2 benzenes (Vitamin L1 and dopamine), 1 organic oxide, nucleoside, nucleotide and analogues (5-methylcytidine) and 2 vitamins (L-ascorbic acid, pantothenic acid).

6. Bioinformatics analysis of the potential biomarkers

In order to accurately and objectively evaluate the rationality of screened biomarkers, and to comprehensively and intuitively reflect the relationship between samples in different groups and the differences of the metabolites in the expression patterns of different samples, the expression amounts of different metabolites in the serum samples of the rats in the YBP500 group and the Model group are subjected to a hierarchical clustering analysis. Generally, when the type, content, and number of screened potential biomarkers are reasonable and accurate, the samples of the same group can appear in the same cluster through clustering. Metabolites appeared in the same cluster often have the same or similar expression patterns, and may be in the same or relatively close reaction process during metabolic processes. Correlation analysis can be used to measure the closeness of significantly different metabolites, and further understand the relationship between metabolites of the rats in the YBP500 group and the Model group during a state change process. Kyoto Encyclopedia of Genes and Genomes (KEGG) is one of the most frequently used databases for the research of metabolic regulation pathways, which is used to express and describe massive metabolic pathways and the interrelationships between various metabolic pathways by generating a specific graphic language. KEGG metabolic pathways enrichment analysis is a data statistic method, which is based on a KEGG pathway as a basic unit, is based on a metabolic pathway involved in a species or closely related species as a main background, is used to analyze and calculate significance level of the degree of the metabolite enrichment of different metabolites in each metabolic pathway by Fisher's precise test, and to rapidly screen metabolic and signal transduction pathways with the greatest (most significant) influence.

In general, the color of bands (different signal pathways) in a KEGG metabolic pathways enrichment analysis figure represents P value of a significant difference, the smaller the P value (P is much less than 0.05), the more significant the metabolic pathway or the degree of pathway enrichment, the more statistical significance. In comparison, the value of the horizontal ordinate in the KEGG metabolic pathways enrichment analysis represents the number of differentially expressed metabolites, which directly reflects the degree of influence of different groups on each pathway in an experimental design. In summary, when the KEGG metabolic pathways enrichment analysis is performed, the above two factors (P value and the number of different metabolites) need to be simultaneously considered. Selecting more interested metabolic or signal transduction pathway, and differentially expressed metabolites that have a significant impact on these pathways to perform subsequent bioinformatics analysis, biological test verification or related mechanisms research has more forward-looking significance. In the disclosure, differentially expressed metabolites of the serum samples of rats in the YBP500 group and the Model group are subjected to KEGG metabolic pathways enrichment analysis by a Fisher's preciese test method, and the results show that important pathways such as Central carbon metabolism in cancer, Protein digestion and absorption, Aminoacyl-tRNA biosynthesis. ABC transporters. Mineral absorption, Bile secretion of ovariectomized osteoporotic rats treated with high-concentration bovine bone peptide (YBP500) significantly change.

7. Potential biomarkers-involved metabolic pathways and regulatory network analysis

Common differential metabolites of the YBP500 group vs. Model group and the Sham group vs. the Model group, include 12 metabolites of erucic amide, (4Z, 7Z,10Z,13Z,16Z,19Z)-4,7,10,13,16,19-docosahexaenoic acid, isoleucine-alanine (Ila-Ala), 1-stearoyl-2-hydroxy-sn-glycerol-3-phosphocholine, DL-indole-3-lactic acid, 4-pyridoxic acid, methylglyoxal, 1-palmitoyl-2-hydroxy-sn-glycerol-3-phosphoethanolamine, pantothenic acid, D-mannose, D-tagatose, and D-fructose. The changes of 4 metabolites of (4Z, 7Z,10Z,13Z,16Z,19Z)-4,7,10,13,16,19-docosahexaenoic acid, 1-stearoyl-2-hydroxy-sn-glycerol-3-phosphocholine, 1-palmitoyl-2-hydroxy-sn-glycerol-3-phosphoethanolamine, and isoleucine-alanine (Ila-Ala) present the same trend in the YBP500 group and the Sham group, and all show an up-regulated trend; and the changes of 5 metabolites of 4-pyridoxic acid, D-mannose, methylglyoxal, D-tagatose, and D-fructose show a down-regulated trend. It is noted that 9 metabolites of 1-stearoyl-2-hydroxy-sn-glycerol-3-phosphocholine, (4Z, 7Z,10Z,13Z,16Z,19Z)-4,7,10,13,16,19-docosahexaenoic acid, 1-palmitoyl-2-hydroxy-sn-glycerol-3-phosphoethanolamine, isoleucine-alanine (Ila-Ala), 4-pyridoxic acid, D-mannose, methylglyoxal, D-tagatose, and D-fructose may be potential biomarkers. 8 common differential metabolites of the ES group vs. Model group and the Sham group vs. the Model group, include 1-stearoyl-sn-glycerol-3-phosphocholine, 1-oleoyl-sn-glycerol-3-phosphocholine, 1-O-(cis-9-octadecenyl)-2-O-acetyl-sn-glycerol-3-phosphocholine, L-palmitoyl, L-pyroglutamic acid, isoleucine-arginine, 1-palmitoyl-sn-glycerol-3-phosphocholine, and pantothenic acid, and the above 8 metabolites have a highly consistent change trend in the ES group and the Sham group. The changes of 6 metabolites of 1-stearoyl-sn-glycerol-3-phosphocholine, 1-oleoyl-sn-glycerol-3-phosphocholine, 1-O-(cis-9-octadecenyl)-2-O-acetyl-sn-glycerol-3-phosphocholine, L-palmitoyl, 1-palmitoyl-sn-glycerol-3-phosphocholine, and pantothenic acid present an up-regulated trend; and the changes of isoleucine-arginine and L-pyroglutamic acid show a down-regulated trend. Besides, 3 common differential metabolites of L-citrulline, pantothenic acid and arachidonic acid of the YBP500 group vs. Model group and the ES group vs. Model group are screened in the disclosure, and the above 3 metabolites have the same change trend between the YBP500 group and Model group, and between the ES group and Model group, which indicates that the two may have a similar mechanism in interfering with bone metabolism in the rats with osteoporosis. In summary, 8 ipids and lipid-like molecules (taurine, arachidonic acid, 1-palmitoyl-2-hydroxy-sn-glycerol-3-phosphoethanolamine, (4Z, 7Z,10Z,13Z,16Z,19Z)-4,7,10,13,16,19-docosahexaenoic acid, 1-stearoyl-2-hydroxy-sn-glycerol-3-phosphocholine, taurodeoxycholic acid, taurochenodeoxycholate and taurocholic acid), 2 organic acids and their derivatives (D-erythro-sphingosine-1-phosphate and L-citrulline), and 1 neurotransmitter (serotonin) are screened in the disclosure, and the above 11 metabolites can be biomarkers in the anti-osteoporosis activity of bone peptide.

By the above signal pathways enrichment analysis of the differential metabolites, the process of bovine bone peptide in the intervention of osteoporosis of the ovariectomized rats has a certain relevance with membrane transport (ABC transports), digestive system (protein digestion and absorption, mineral absorption), translation (aminoacyl-tRNA biosynthesis), amino acid metabolism (arginine and proline metabolism, valine, leucine and isoleucine degradation), lipid metabolism (bile secretion, primary bile acid biosynthesis, taurine and hypo taurine metabolism), cellular immunity, nervous system, carbon metabolism and endocrine system. The skeleton of an organism are very active metabolic tissues, which maintains a constant bone mass by continuously removing old bone and synthesizing new bone. In a bone remodeling process, lipid metabolism plays a vital role. A large amount of evidence has shown that there is a close relationship between bone mass and bone marrow fat content. The research proportion of bone lipid metabolism in the field of bone metabolism is increasing. Fatty acids, phospholipids and endogenous lipid metabolites have been proved to be related to the key signal transduction of osteoblast proliferation, differentiation and bone mineralization. The disclosure screens biomarkers in the anti-osteoporosis activity of bone peptide based on UPLC/Q-TOF-MS combined non-targeted metabolomics methods, further clarifies its metabolic pathways and regulatory networks, and comprehensively, efficiently and systematically evaluates the mechanism of the anti-osteoporosis activity of bone peptide from the overall level, provides an exemplary research for activity and function evaluation of bone peptide and screening of biomarkers of the anti-osteoporosis activity, and provides theoretical support for the development of bone peptide products with biological activity.

In-depth analysis of the changes in serum metabolic patterns of the ovariectomized rats with osteoporosis after intervention in different groups can help to further reveal the metabolic reorganization mechanism after intervention of bovine bone peptide. 11 significantly up-regulated or down-regulated endogenous metabolites including 8 kinds of lipid and lipid-like molecule, 2 kinds of organic acids and their derivatives, and 1 kind of neurotransmitter are identified as potential biomarkers in the intervention therapy of bovine bone peptide in the disclosure. It can be seen that, as a key organ for the metabolism of sugars, amino acids, lipids and bile acids, the liver metabolic pathways related to these nutrients in rats with osteoporosis have undergone extensive changes. The bovine bone peptide intervention group can significantly reverse the abnormal metabolism of rats with osteoporosis, which supports the therapeutic effect of bovine bone peptide on ovariectomized rats. KEGG pathway analysis shows that ovariectomy can significantly change the endogenous metabolites of rats and induce metabolic disorders. Bovine bone peptide mainly balances metabolic disorders by intervening in amino acid metabolism and lipid metabolism (especially unsaturated fatty acid metabolism). Related pathway regulation networks are shown in FIG. 6. In summary, 11 screened biomarkers in the anti-osteoporosis activity of bone peptide can be used to better predict and evaluate anti-osteoporosis activity of polypeptides. The disclosure provides an exemplary research for the activity and function evaluation of natural products (polypeptides), and provides theoretical support for the systematic evaluation of the anti-osteoporosis activity of bone peptide and the development of bone peptide products with biological activity.

The number of modules and the processing scale described here are used to simplify the description of the present disclosure. The application, modification and change of the biomarker in osteoporosis intervention therapy by bone peptide, screening method and its use in the present disclosure are obvious to those skilled in the art.

As mentioned above, in order to clarify the protection or recovery mechanism of bone peptide on osteoporosis, the disclosure systematically evaluates the anti-osteoporotic activity of bone peptide based on an automatic serum biochemical analysis, a three-point bending test method, a Micro-CT method, a H&E staining method, and UPLC/Q-TOF-MS combined non-targeted metabolomics methods; performs discriminant analysis to identify and screen significantly different metabolites (biomarkers) by serum metabolic fingerprints, provides basic data for systematic evaluation of the anti-osteoporotic activity of bone peptide, and provides theoretical support for the development of bone peptide products with biological activity.

Although the embodiments of the disclosure have been disclosed above, they are not limited to the applications previously mentioned in the specification and embodiments and can be applied in various fields suitable for the disclosure. For an ordinary skilled person in the field, other changes may be easily achieved. Therefore, without departing the general concept defined by the claims and their equivalents, the disclosure is not limited to particular details and embodiments shown and described herein.

What is claimed is:

1. A biomarker in osteoporosis intervention therapy by bone peptide, the biomarker comprising a lipid and lipid-like molecule, an organic acid and its derivative, and/or a neurotransmitter, wherein the lipid and lipid-like molecule comprises one or more of taurine, arachidonic acid, 1-palmitoyl-2-hydroxy-sn-glycerol-3-phosphoethanolamine, (4Z, 7Z,10Z,13Z,16Z,19Z)-4,7,10,13,16,19-docosahexaenoic acid, 1-stearoyl-2-hydroxy-sn-glycerol-3-phosphocholine, taurodeoxycholic acid, taurochenodeoxycholate or taurocholic acid.

2. The biomarker in osteoporosis intervention therapy by bone peptide according to claim 1, wherein the organic acid and its derivative comprise D-erythro-sphingosine-1-phosphoric acid and/or L-citrulline.

3. The biomarker in osteoporosis intervention therapy by bone peptide according to claim 1, wherein the neurotransmitters is serotonin.

4. A screening method of a biomarker in the anti-osteoporosis activity of bone peptide, comprising the following steps:
step one, collecting samples: collecting bone tissues and serum samples from animals treated with bone peptide, wherein the bone tissues comprise left femurs, right femurs and right tibias;
step two, determining a content of a serum bone turnover marker by an automatic serum biochemical analyzer, and analyzing the effect of the bone peptide on the content of the serum bone turnover marker;
step three, determining biomechanical indexes of the left femurs by a three-point bending test method, and analyzing the effect of the bone peptide on mechanical indexes of femurs;
step four, determining biomechanical indexes of the right femurs by a Micro-CT method, and analyzing the effect of the bone peptide on morphologically mechanical indexes of femurs;
step five, determining bone microstructure indexes of the right tibias by a H&E staining method, and analyzing the effect of the bone peptide on bone microstructures of tibias of rats;
step six, systematically screening and analyzing a differential biomarker in the anti-osteoporosis activity of the bone peptide, as well as its metabolic pathways and regulatory networks based on a non-targeted metabolomics method.

5. The screening method of a biomarker in the anti-osteoporosis activity of bone peptide according to claim 4, wherein the serum bone turnover marker comprises bone gamma-carboxyglutamic acid containing proteins, bone alkaline phosphatase, procollagen type I N-peptide, tartrate-resistant acid phosphatase, serum C-terminal telopeptide of type I collagen, and urinary deoxypyridinoline;
the mechanical indexes comprise fracture load, elastic load, elastic deformation, bending energy and stiffness coefficient of bone; and
the morphologically mechanical indexes comprise trabecular bone density, bone volume fraction, trabecular bone spacing, trabecular bone thickness, trabecular bone number, and cortical bone thickness.

6. The screening method of a biomarker in the anti-osteoporosis activity of bone peptide according to claim 4, wherein the animals are rats.

7. The screening method of a biomarker in the anti-osteoporosis activity of bone peptide according to claim 4, in the step one, a treatment process of the animals treated with the bone peptide comprising perfusing an animal with an bone peptide solution, wherein a concentration of the bone peptide solution is 100 mg/kg, 200 mg/kg or 500 mg/kg according to the weight of the animal.

8. The screening method of a biomarker in the anti-osteoporosis activity of bone peptide according to claim 4, in the step one, the treatment process of the animals treated with bone peptide further comprising automatically collecting urine of the animals with a metabolic cage, wherein the metabolic cage comprises a cage body with a bottom and a metabolite collecting part; the metabolite collecting part being arranged below the cage body and comprising a barrel and a cover mounted on an upper end of a peripheral wall of a first side of the barrel, an upper end of a peripheral wall of a second side of the barrel being provided with a drainage port, a solid-liquid separating part being arranged in the barrel, the solid-liquid separating part comprising an arc-shaped partition plate with a first end fixed with a peripheral wall of the barrel and multi-stage filter plates, which divide an inner space of the barrel into a first accommodating space and a second accommodating space, the multi-stage filter plates being arranged in the second accommodating space along a vertical direction, and the multi-stage filter plates being successively arranged end to end to form a folded-line diversion channel, a depth of a bottom wall of the barrel from the first side to the second side becoming larger, the cover comprising an upper edge bent upwards; a first part of the cover connected to a the barrel being provided with a first through hole, the first end of the arc-shaped partition plate being provided with a second through hole, the second through hole being provided with a filter membrane with 5-20 μm pore size, and the multi-stage filter plates being provided with filter pores whose pore sizes becoming smaller and smaller along the vertical direction from top to bottom and all being larger than the pore size of the filter membrane in the second through hole.

9. The screening method of a biomarker in the anti-osteoporosis activity of bone peptide according to claim 4, wherein the bone peptide comprises the following peptides: amino acid sequences shown as SEQ ID NO 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58 and 59.

10. A use of the biomarker according to claim 1 in scientific research, and intervention therapy or diagnosis of osteoporosis.
